(19) United States
(12) Patent Application Publication
(10) Pub. No.: US 2024/0157158 A1
(43) Pub. Date: May 16, 2024

(54) NEUROMODULATION DEVICE PROGRAMMING OPTIMIZING METHOD AND SYSTEM

(71) Applicant: INBRAIN NEUROELECTRONICS S.L., Barcelona (ES)

(72) Inventors: Aina Maull MIQUEL, Barcelona (ES); Egbertus J.M BAKKER, Wijk en Aalburg (NL); Carolina AGUILAR, Villars sous Yens (CH); Marina Saiz AIRA, Las Rozas de Madrid (ES)

(21) Appl. No.: 18/548,473

(22) PCT Filed: Feb. 28, 2022
(86) PCT No.: PCT/EP2022/054980

§ 371 Date: Aug. 30, 2023

(30) Foreign Application Priority Data
Mar. 1, 2021 (EP) 21160093.7

(51) Int. Cl. A61N 1/372; A61N 1/36; G06F 3/01; G06F 3/16

(52) U.S. Cl. CPC A61N 1/37247; A61N 1/36132; A61N 1/36139; G06F 3/017; G06F 3/167

(57) ABSTRACT

The present invention relates to a method for programming a neuromodulation device (100), comprising:
(a) determining an initial health condition of a patient;
(b) determining one or more conditions and/or symptoms and/or expected outcomes for the patient;
(c) determining initial settings for the neuromodulation device including one more treatment parameters based on the determined initial health condition of the patient and/or the one or more conditions and/or symptoms and/or expected outcomes, and delivering stimulation to the patient based on the determined initial settings;
(d) determining a patient's response with respect to the delivered stimulation
(e) implementing an algorithm that is set to modify, based on the determined patient response, the one or more treatment parameters within a predefined range and determine new settings for the neuromodulation device;
(f) repeating steps (d) and (e), and
(g) maintaining the one or more treatment parameters settings identified as optimal by the algorithm.

### TECHNICAL FIELD

The present invention relates to a method and system for programming a neuromodulation device. More specifically, the present invention provides a method and system for optimizing programming of a neuromodulation device.

### BACKGROUND AND SUMMARY

Programming operations for neuroelectronic treatment or a neuromodulation device, e.g., a DBS stimulator, are overall complicated.

Moreover, with the development of new or plural treatment options (e.g., burst modes, intermitted stim, directional stimulation, increased number of electrodes or the like), programming operations became even more complicated and time consuming.

Finding the right settings for a specific kind of treatment and patient is crucial to the quality of the treatment outcome.

In the state of the art, some tooling is available to assist the operator, e.g., in visualizing targets and clustering of parameters.

Nevertheless, while programming the neuroelectronic treatment or the neuromodulation device, the operator (e.g., a physician or other clinical staff) is still requested to operate a selection of the settings based on indications and his/her own experience.

Thus, finding the optimal settings for the specific treatment and patient remains difficult and time-consuming

In the state of the art, finding the optimal treatment parameters settings requires an iterative process where the patient is requested to return to the hospital several times, this involving a significant amount of time and clinical resources. This further determines a major burden on the patient, since this iterative process cannot be implemented in a home environment but, to the contrary, needs to be carried out in a hospital structure.

The patient may be allowed to alter one or more parameters to some degree, e.g., by using patient a programmer such as a remote control or the like.

In this way, the patient is enabled to implement parameter variations within a certain predefined range (e.g., plus or minus 0.2V on a 3V stimulation voltage setting), which allows obtaining a finer tuning of the baseline parameter settings. The predefined range may vary per country and/or hospital policies.

In case a neuromodulator device allows the implementation of more than one program, a further option that available to the patient is to operate a selection among a plurality of programs having different therapy settings.

For instance, different programs can be defined for corresponding conditions, e.g., walking, speaking or the like. Accordingly, the patient can select a specific program depending on his/her activity or mood, e.g., by using a remote control. In other words, the patient is able and/or given the possibility to adjust and fine tune the treatment parameters to better adapt to the needs for a particular activity. Then, this predefined set of settings can be stored as programs. One of the objectives of this feature is optimizing the neuromodulation effect for a given activity improving the patient's experience rather than optimize the therapy in general.

However, the possibility of selecting among different programs represents more an optimization of the conditions rather than an optimization of the treatment itself.

Further, every program needs its own specific optimizing steps, which is again a difficult and time-consuming operation.

In the state of the art, when optimized settings are defined for the patient, said setting are then used all day/every day.

Then, in case the identified optimized settings are no longer efficient or no longer meet the patient's needs and preferences, the patient may select among extra programs, e.g., through a remote control. This allows the patient to switch between predefined programs depending on his/her current conditions.

Nevertheless, even with the provision of said extra programs, this known method still configures a ‘one size fits all’ solution, and not a therapeutic approach that is focused on the patient's current symptoms, needs and priorities.

In this regard, it is noted that the patient's condition and wishes can considerably vary depending on the time of day and the medication effectiveness at those times of the day.

Apparently, this aspect cannot be properly considered by using a discrete set of programs.

Several scientific articles have emphasized not only the complexity of programming operations but also the fact that, presently, the operators program the neuromodulation device based on the patient's motor symptoms and not on the outcome that actually matters to the patient.

Progress in developing new neurostimulation indications may be also hampered by the fact that some brain conditions (e.g., some forms of dystonia, depression, anxiety or addiction syndromes) can take several weeks to show a stimulation response.

As mentioned in the foregoing, determining the right therapy settings is crucial to the quality and efficiency of the treatment.

In the state of the art, all settings of neuromodulator devices are essentially determined by the operator, e.g., a physician or other clinical staff. That is, the selection of the settings is essentially based on the operator's knowledge, further supported by additional inputs of different kind (e.g., the patient's feedback or response to pharmacological treatment).

As a further support, information from other procedures or by tooling making abstractions of complex setting methodologies may also be used.

Nevertheless, the operator still remains the figure that actually defines the treatment parameters settings.

In his book Redefining Health Care, Harvard Business Press, 2006, Harvard Professor Michal Porter described Value Based Healthcare by putting the patient at the center and defining healthcare value with a simple equation:

$$\\ Value = \frac{Outcomes that matter to patients}{Cost}$$

Placing the patient, his/her priorities and quality of life, in the center while making decisions regarding treatment parameters settings turned out to be beneficial to the efficacy of treatment.

Specifically, through this approach, patients are empowered to prioritize and take control over their disease in a way that can help the system to provide additional value to the individual and the society in the long term.

Several scientific publications (cf., e.g., Schtipbach M. et al, Neurosurgery in Parkinson's Disease: a distressed mind in a repaired body?, in Neurology Jun. 27, 2006, 66(12):1811-6) draw the attention on situations where, although motor symptoms of the patients were satisfactorily fixed, nevertheless the quality of life of the patient still remained poor.

The above specified patient-centered approach enables to largely avoid the occurrence of unsatisfactory situations of this kind.

As an example, non-motor symptoms in Parkinson Disease patients are independently associated with worse quality of life, although most of the patients have better control of their motor symptoms. Thus, understanding and taking into proper account patterns and effects related to non-motor symptoms of the disease would be significant in order to define a proper treatment strategy allowing to fulfill the current needs and priorities of the patient.

Nevertheless, in practice, these aspects have been frequently unattended.

An aim of the present invention is to provide a method that allows optimizing programming of a neuromodulation device to more efficiently obtain a desired outcome for a patient.

Another aim of the invention is to provide a method as above specified where adaptation of treatment parameter settings can be easily carried out in a home environment, without requesting the intervention of a physician or other clinical staff.

Yet another aim of the invention is to provide a method as above specified that allows the patient to easily and intuitively provide a feedback on one or more treatment parameter.

It is also an aim of the present invention to provide a system for carrying out the above specified method.

According to the invention, these aims are achieved by means of a method for programming a neuromodulator device as described herein.

Advantageous embodiments of the present invention regarding the method are described herein.

Specifically, the present invention provides a method for programming a neuromodulation device comprising:
(a) determining an initial health condition of a patient;
(b) determining one or more conditions and/or symptoms and/or expected outcomes for the patient
(c) determining initial settings for the neuromodulation device including one or more treatment parameters, based on the determined initial health condition of the patient and the one or more conditions and/or symptoms and/or expected outcomes, and delivering stimulation to the patient based on the determined initial settings;
(d) determining a patient's response with respect to the delivered stimulation;
(e) implementing an algorithm that is set to modify, based on the determined patient's response, the one or more treatment parameters within a predefined range and determine new settings for the neuromodulation device;
(f) repeating steps (d) and (e) at predefined time intervals and/or in response to a user input until obtaining one or more treatment parameters settings that are identified as optimal by the algorithm, based on the patient's response to stimulation, and
(g) maintaining the one or more treatment parameters settings identified as optimal by the algorithm and optionally monitoring the patient to detect the occurrence of deviations in real time.

The invention is based on the basic idea that, by placing the patient and his/her symptoms and priorities in the center while making decisions regarding treatment parameters settings, the therapeutic outcome can be optimized and better satisfy the actual needs and priorities of the patient. Furthermore, the adaptation of the treatment parameters settings can be easily carried out in a home environment, without the need for the patient to visit a hospital and/or the intervention of qualified clinical staff.

As mentioned, according to the state of the art the finding of the optimal treatment parameters settings requires an iterative process where the patient is requested to return to the hospital several times, this involving a significant amount of time and clinical resources. This further determines a major burden on the patient, since this iterative process cannot be implemented in a home environment but, to the contrary, needs to be carried out in a hospital structure. This increases the burden of the patient but also reduces considerably the data available for such treatment optimization. According to the invention, however, this problem is solved: Getting data from home environment (through e.g. apps such as mhealth/digital solutions) and from earlier in the process (even in the time patient has no implant yet), provides better optimization as it allows to cluster patients and select initial parameters, lowering the dimensions of the problem and later tune them, according to the disease progression/symptoms fluctuations, that are tracked using the method for programming a neuromodulation device.

The method and its steps can be realized, either partially or completely by means of a software module, for example an app or the like.

In particular, in step (g) a patient's database can be used to which the individual initial health and preferences (symptoms and expected outcomes) can be compared to, in order to compute the optimal initial settings.

From steps (a) to (g) there are two iterative processes that are overlapped in the text.

As a first step there is a data integration, i.e. in step (a) an initial health condition and the diagnostic that the doctors have provided is determined and in step (b) determining one or more conditions and/or symptoms and/or expected outcomes for the patient

Once the data were aggregated then one can move to the following step:

The expected output of this first data integration will be the definition of a suitable treatment among a preestablished set of defined treatments.

Inside of a treatment (see also e.g. FIG. 4-6), in particular in steps (d), (e) and (f)) are correct in the sense that the treatment parameters (better defined as variables) that can be modified are optimized to refine the default treatment that was linked to the patient initially. This way, a control system by decreasing the difference between patient's response and feedback can be provided.

The patient's initial health condition can be determined by a physician or other clinical staff, e.g., based on the patient's anamnesis or a questionnaire administered to the patient.

For instance, the patient may be affected by Parkinson's disease.

The algorithm is set to modify the treatment parameters in a predefined range within which the different parameter values are considered safe (e.g., there is no risk of tissue damage).

Advantageously, the patient's response with respect to the delivered stimulation can be determined based on a patient's feedback on the one or more treatment parameters provided through a user input device.

Additionally or alternatively, the method may comprise recording brain signals of the patient during stimulation delivery.

The patient's response with respect to the delivered stimulation can be determined based on the recorded brain signals.

In particular, the method can comprise the following steps:
recording brain signals of the patient during stimulation,
the patient's (P) response with respect to the delivered stimulation is determined based on the recorded brain signals.

This way, real patient data can be used to optimize the treatment.

Furthermore, the method can further comprise the following step of detecting one or more movements and/or gestures of the patient during stimulation delivery.

By detecting such data, the correctness of the delivered stimulation can be checked and also side effects can be detected. Based on this, e.g. a finetuning of the stimulation can be done.

A specific biomarker may also be used as a possible user input.

For instance, an action of clicking with the tongue can be implemented by the patient to provide a feedback.

As an example, one click can be used to provide the feedback “I am feeling well”, while two clicks may be used to provide the feedback “I am feeling bad”.

Tongue clicking can be detected, e.g., through a sensor that is embedded in a head-mounted IPG.

Additionally or alternatively, the method may comprise detecting one or more movements and/or gestures of the patient during stimulation.

The patient's response with respect to the delivered stimulation can be determined based on the detected one or more movements and/or gestures of the patient during stimulation delivery.

The one or more movements and/or gestures of the patient may be detected through sensor means such as an accelerometer, a gyroscope, an EMG recorder and/or camera. Also voice commands or other suitable commands can be used.

In particular, said sensor means and/or camera can be included in a portable, optionally wearable, device of the patient (e.g., as a smartphone, a smartwatch or the like). Advantageously, the method may further comprise organizing the determined one or more conditions and/or symptoms and/or expected outcomes for the patient according to predefined criteria of priority.

Said criteria of priority can be defined by the patient, a clinician or other medical staff, or can be automatically set.

Accordingly, the one or more conditions and/or symptoms and/or the expected outcomes can be listed and prioritized in order of importance, e.g., based on the standardized and validated set of outcomes by disease defined by by ICHOM (International Consortium of Health Outcomes Measurements).

The initial settings for the neuromodulation device may be determined based on collected data from one or more groups of patients having identical or similar health condition than the patient subject to treatment.

This enhances the possibilities of finding the best settings for the patient subject to treatment.

In the method of the invention, the one or more treatment parameters may include one or more of:

neuromodulation frequency ranges;
neuromodulation current ranges;
electrode selection;
burst modes and patterns;
level of patient activity in “on” and “off” conditions;
pharmacology;
sleep patterns;
mood patterns;
environmental and/or weather conditions that may affect a health condition of the patient, and/or
one or more anticipated activities of the patient.

The level of patient activity in “on” and “off” conditions is to be understood as follows: ‘On’ time is when the pharmacological treatment e.g. with Levodopa (in case of Parkinson's disease) is working well and the patient symptoms are controlled. ‘Off’ time is when Levodopa is no longer working well and symptoms such as tremor, rigidity and slow movement re-emerge.

The above mentioned parameters are not listing all relevant parameters, there may be further parameters. Basically, all parameters, which do have an effect on the treatment outcome can be taken into account.

Neuromodulation frequency ranges may be usual ones for stimulation brain structures and/or other neural structures, e.g. in the range from 0-150 Hz.

Current ranges can be selected in the ranges as currently and commonly used for neuro stimulation.

Electrode selection does include the electrode design and arrangement, but also other parameters relating to the arrangement and/or placement and/or design of the electrodes.

Burst modes and patterns can be chosen as currently and commonly used for neuro stimulation.

Pharmacology means the use of pharmacological agents for treating the disease of the patient.

Sleep patterns and/or mood patterns of the patient and/or environmental and/or weather conditions that may affect a health condition of the patient also play a role and give useful information about the status and progression of the disease of the patient. Taking them into account increases the accuracy of the treatment plan.

Anticipated activities of the patient can also be taken into account as this might also affect therapy planning and the therapy goals. Keeping a patient for example completely active might require other treatment approaches when compared to an approach, where a patient just should stay mobilized with partial assistance of nurses etc. available.

Advantageously, the user input device may be a portable or wearable device.

In particular, the user input device may be a smart device (e.g. a smartwatch).

The user input device, preferably portable or wearable smart device, may include a touch-sensitive display that is configured and adapted to display a user interface including a plurality of selectable options, and allow selection of one or more of the displayed selectable options through a touch input (e.g., tapping, pushing or squeezing).

The selectable options can be displayed on the touch-sensitive display at predefined time intervals (e.g., every half an hour).

Additionally or alternatively, a questionnaire may be displayed on the touch-sensitive display at predefined time intervals, asking for the patient's feedback on one or more specific questions.

Additionally or alternatively, a pre-programmed voice command can be provided to periodically ask for the patient's feedback.

The displayed plurality of selectable options may be color-coded.

For instance, green color can be associated to the feedback “I am feeling well”, yellow color can be associated to the feedback “I am feeling neutral”, and red color can be associated to the feedback “I am feeling bad”.

The selectable options can be presented to the user in the form of selectable icons, preferably selectable color-coded icons.

This enables the patient to easily an intuitively provide his/her feedback on the one or more treatment parameters.

Additionally or alternatively, the user input device may further include means for receiving a vocal input or voice input from the patient (e.g., a microphone).

In this case, the patient may provide his/her feedback through a vocal input, e.g., by saying “I am feeling well”, “I am feeling neutral”, or “I am feeling bad”.

Advantageously, the method of the invention may further comprise:
collecting information on environmental and/or physiological conditions one or more movements and/or gestures of the patient in real time, and
processing the collected information for use by the algorithm in determining the new settings for the neuromodulation device.

Accordingly, the new settings for the neuromodulation device can be more accurately defined by the algorithm.

The environmental condition(s) of the patient may include day/night condition, active/inactive condition, weather conditions or the like.

The physiological condition(s) may include heart rate, laying/standing/sitting/sleeping condition or the like.

Said information on environmental and/or physiological conditions and/or one or more movements and/or gestures of the patient of the patient may be collected through sensor means and/or a camera.

Said sensor means and/or camera may be embedded in a portable device, preferably a smart device, of the patient (e.g., a smartphone or the like).

Advantageously, the method may further comprise:
storing, on a database, preferably a cloud-based database, data regarding different groups of patients, each of the groups including patients having similar or identical health and therapy conditions;
processing said data according to predefined criteria, and
defining the initial settings of the neuromodulation device and the algorithm based on data of a group of patients having similar or identical health and therapy conditions than the patient subject to treatment.

Accordingly, accuracy in defining the initial settings as well as setting the algorithm can be improved and the treatment can be further optimized.

The method may further include setting one or more filters in said database.

The provision of said one or more filters allows the operator to more easily find a group of patients having similar or identical health and therapy conditions than the patient subject to treatment, based on the determined initial health condition of the patient and/or the determined one or more conditions and/or symptoms and/or expected outcomes for the patient.

In particular, by inputting information relating to the determined initial health condition of the patient and/or the determined one or more conditions and/or symptoms and/or expected outcomes for the patient into the database (e.g., through a keyboard or the like), the operator is allowed to more easily and reliably find a group of patients having similar or identical health and therapy conditions than the patient subject to treatment. Advantageously, the algorithm may be further set to predict disease symptoms and/or fluctuations for the patient over time based on the determined patient's response to the delivered stimulation.

The symptoms may be motor symptoms.

Additionally or alternatively, the symptoms may be non-motor symptoms.

In particular, the algorithm may be set to predict disease fluctuations over a short temporal scale, e.g., within a day.

By predicting short-term disease fluctuations it is possible to provide an immediate feedback to the patient, thereby enhancing the patient's engagement and allowing the patient to better plan his/her daily routine.

Additionally or alternatively, the algorithm may be set to predict disease progression over a longer temporal scale.

This can mean that the algorithm might predict the on and off states of the patient and recommend specific actions to stay in on as much as possible. But also might predict other degenerative episodes such as dysarthria, cognitive deterioration of even potential falls. by preventing this over this longer temporal stage we can avoid cost of the healthcare system. Longer temporal stage might mean, deterioration capacity prediction over the next 3-5 years of patient's life.

In particular, this can be done as follows:

The patient's initial health condition and chosen patient data are inputted into an extensive patient database. The database includes filters that help the doctor to tailor the population used to find an initial treatment.

The treatment personalization can be based on clusterization of disease stages using population databases and patient profiles.

For example, the following kinds of patient profiles can be (inter alia but not limited to) used:

Controllers: needed data to manage their own disease. Health is their focus.

Warriors: Disease needs submission and the want to live their life to the fullest.

Easy Going: Live a life as normal as possible, they are not so interested in data if its interjecting with their normality.

Hostage: Pray of the disease, moody, instable.

Defeated: Ruled by the disease, survival mode.

The disease parametrization distinguishes between two time-scales. There can be a shorter-term fluctuation over daily symptoms (both motor & and non-motor and there can be a longer term-disease progression, describing wearing-off and changes on time on/off ranges and classifying patient into different patient data (PD) stages (early, moderate and advanced PD). The idea behind the prediction of disease progression over a longer temporal scale is to be capable to evaluate the different output variables and feedback from the patient to understand in which state of severity is this patient and to predict the tendencies to a different disease/severity state.

This allows obtaining an optimized treatment.

Advantageously, the algorithm may be further set to predict disease degeneration for the patient based on detected deviations over time.

This allows to detect earlier signs of key changes relating to the patient's disease (such as Parkinson's disease).

The detected deviations may be analyzed with respect to the patient's anamnesis and/or with respect to data regarding a group of patients having similar or identical health and therapy conditions, stored in the database.

In particular, this can be done as follows:

By means of patient monitoring sensors and active symptom reporting app (i.e. for example the INHEALTH app) which is tailored to individual needs (taking of those outcomes and variables that matter to a particular patient), PD individual ups and downs are predicted (including both either shorter scale symptoms fluctuations over a predefined and selected treatment (Tn) and/or disease progression over longer temporal scales)

The ups and downs prediction is focused mainly in short term fluctuations and based in forecast prediction and extraction of individual patterns. The objective is to generate an immediate feedback to the patient, increase patient engagement and empower them. Similar to when a person checks the forecast to better plan his/her routine the patient feedback is not only used to optimize therapy but to generate an individual report of key metrics and a tool to improve the management of the disease. The symptom's deviation detection is done by analyzing over specified thresholds. A predictive maintenance of device is possible.

This allows better understanding the causes that generate the detected deviations.

Further, it is possible to adjust neurostimulations setting to enhance treatment effectiveness and prevent disease degeneration.

For instance, it may be determined whether the detected deviations are related to disease progression and, if so, timely adjust treatment parameters to prevent further degeneration.

In addition, it is possible to determine whether there is a need for delivery of earlier neuro stimulation treatment (i.e., before refractory symptoms become apparent), to improve patient outcomes and confer long-term symptomatic benefit.

In particular, control algorithms allow to optimize treatment, to control fluctuations and to extract hidden patterns that may provide earlier signs of key changes within disease. If some important deviations are observed in time, they are studied against the historic data from the patient together with the population data base (DB) to understand the origin and the best solution. This allows to see if changes are a sign of illness progression and if there is the chance to change the stablished treatment before the symptoms get worst thus preventing patient degeneration.

A study of the repetitive deviation can be performed. Automatic control over abnormal symptom fluctuation, alerting physician of a change either originating from altered patient routine or informing that a change of prescribed treatment is required

There is a need for earlier neurostimulation therapy as an earlier use of DBS confers long-term symptomatic benefit. Initial patient monitoring data from e.g. an app such as the INHEALTH app can recommend physician to prescribe neuromodulation therapy before refractory symptoms are obvious, improving patient outcomes.

A turning point to change a stablished treatment can be defined to predict illness degeneration.

With time, the algorithm can be trained to make predictions that the patient can use to improve his/her health outcomes.

For example, the algorithm might predict when the patient may encounter the best conditions to perform a predefined activity (e.g., go for a long walk), and if the weather that day is going to be adequate to actually perform it, so that the patient can better plan better his/her activities to obtain the best outcome.

The method of the invention may further comprise determining, through the optimization algorithm, that the patient is in need of hospital assistance upon detecting a negative feedback from the patient over a predetermined time period. On the other hand, patients having a normal condition (e.g., showing a positive feedback over a predetermined time period) are allowed to continue the treatment in their home environment and only visit the hospital for prescribed checkups (e.g., on an annual basis).

This allows for better allocation and optimization of healthcare resources and organization of clinical staff.

Optionally, the method may further include triggering an alert to clinical staff notifying that the patient is in the need of hospital assistance.

The invention further provides a system for programming a neuromodulation device, said system comprising:
a neuromodulator device, and
processing module.

Specifically, the processing module of the system is configured and arranged to:
(a) determine an initial health condition of the patient;
(b) determine one or more conditions and/or symptoms and/or expected outcomes for the patient;
(c) determine initial settings for the neuromodulation device including one more treatment parameters, based on the determined initial health condition of the patient and the one or more conditions and/or symptoms and/or expected outcomes, and deliver stimulation to the patient based on the determined initial settings;
(d) determine a patient's response with respect to the delivered stimulation;
(e) implement an algorithm that is set to modify, based on the determined patient's response, the one or more treatment parameters within a predefined range and determine new settings for the neuromodulation device;
(f) repeat steps (d) and (e) at predefined time intervals and/or in response to a user input until obtaining one or more treatment parameters settings that are identified as optimal by the algorithm, based on the patient's response to stimulation, and
(g) maintain the one or more treatment parameters settings identified as optimal by the algorithm and optionally monitor the patient to detect the occurrence of deviations in real time.

Advantageously, the system may comprise a user input device, configured and adapted to allow a patient to provide a feedback on one or more treatment parameters.

The processing module may be configured and adapted receive and process the patient's feedback on the one or more treatment parameters provided by the patient through the user input device.

Advantageously, the user input device may be a portable or wearable device, preferably a smart device (e.g., a smartwatch).

The user input device may include a touch-sensitive display that is configured and adapted to display a user interface including a plurality of selectable options (e.g., in the form of a list or a population of icons), and allow selection of one or more of the displayed selectable options through a touch input (e.g., tapping, pushing or squeezing).

The system may further comprise a database, preferably a cloud-based database, for storing data regarding groups of patients, each group including patients having similar or identical health and/or therapy conditions.

Advantageously, the system may be operatively connected to means for remotely controlling one or more activities of the patient, thereby allowing to assist the patient from a remote position.

Further details and advantages of the present invention shall now be disclosed in the connection with the drawings.

### BRIEF DESCRIPTION OF THE FIGURES

It is shown in

FIG. 1 a schematic view of an example embodiment of the method and the system according to the present invention with a user input device, specifically a smartwatch, including a touch-sensitive display displaying a plurality of selectable icons;

FIG. 2 a block diagram schematically illustrating a method for programming a neuromodulation device according to one embodiment of the present invention;

FIG. 3 a block diagram schematically illustrating a method for programming a neuromodulation device according to a further embodiment of the present invention;

FIG. 4 a flowchart of a possible embodiment of the method according to the present invention carried out with the system as shown in FIG. 1-3;

FIG. 5 a flowchart of a Patient-centered method for optimizing Parkinson's disease (PD) neuromodulation device programing and for improving patient journey and healthcare resources; and

FIG. 6 a flowchart of a possible application of the method and system according to the present invention.

### DETAILED DESCRIPTION

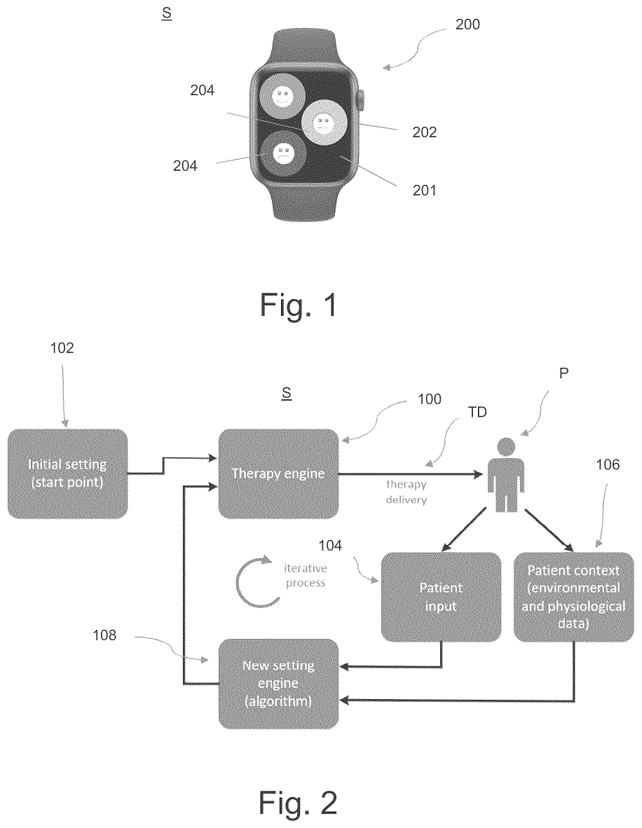

FIG. 1 shows a smartwatch 200 acting as a user input device of a possible embodiment of the present invention, with which a possible embodiment of the method according to the present invention can be carried out.

The smartwatch 200 includes a touch-sensitive display 201 on which a plurality of selectable options 202 is shown.

In the illustrated example, the selectable options 202 are shown as a population of selectable icons 204, each visually showing a respective feeling.

Specifically, a “happy face” is used for the feedback “I am feeling well”, a “neutral face” is used for the feedback “I am feeling neutral”, and a “sad face” is used for the feedback “I am feeling bad”.

Optionally, the selectable options 202 can be color-coded.

FIG. 2 shows a block diagram schematically illustrating a method for programming a neuromodulation device 100 according to an embodiment of the present invention.

Generally speaking, there is an initial setting starting point 102.

This starting point/initial setting 102 is provided to the therapy engine of the neuromodulation device 100.

The neuromodulation device is providing a therapy delivery TD to the patient P.

The patient P can circle back further data to the neuromodulation device 100 as follows:

There can be patient input 104 and also data regarding the patient context 106, such as environmental and physiological data.

The patient input 104 and also the data regarding the patient context 106 will be entered into an algorithm, i.e. a new setting engine 108.

The new setting engine 108 provides further and adjusted setting data to the therapy engine of the neuromodulation device 100.

This process is an iterative process.

As shown in FIG. 2, the method of the invention provides a patient-centered method for programming a neuromodulator device, comprising:
(a) determining an initial health condition of a patient;
(b) determining one or more conditions and/or symptoms and/or expected outcomes for the patient;
(c) determining initial settings for the neuromodulation device 100 including one more treatment parameters, based on the determined initial health condition of the patient and the one or more conditions and/or symptoms and/or expected outcomes, and deliver stimulation to the patient based on the determined initial settings;
(d) determine a patient's response with respect to the delivered stimulation;
(e) implementing an algorithm that is set to modify, based on the determined patient's response, the one or more treatment parameters within a predefined range and determine new settings for the neuromodulation device 100;
(f) repeating steps (d) and (e) at predefined time intervals and/or in response to a user input until obtaining one or more treatment parameters settings that are identified as optimal by the algorithm, based on the patient's response to stimulation, and
(g) maintaining the treatment parameters settings identified as optimal by the algorithm, and optionally monitoring the patient to detect the occurrence of deviations in real time.

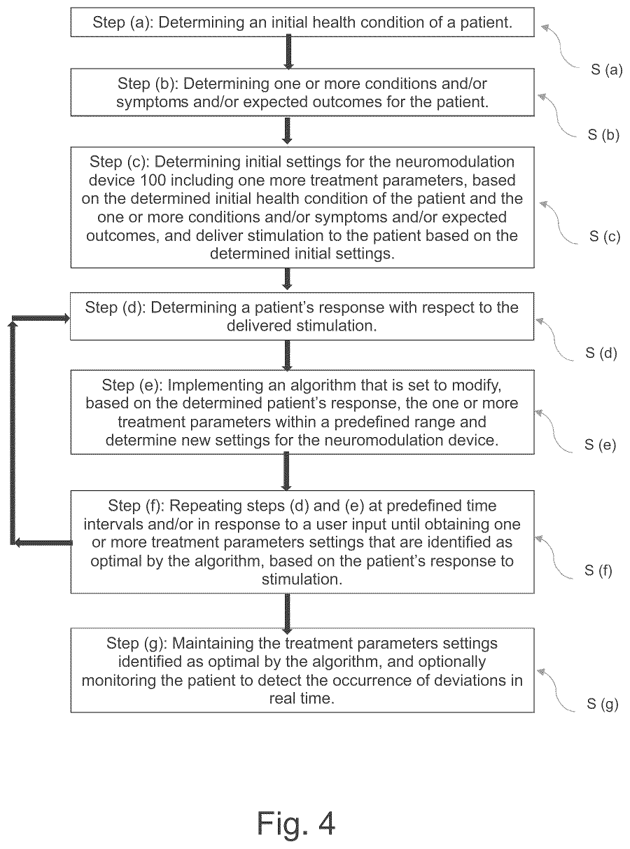

These steps are also shown in the flowchart in FIG. 4, which in particular shows a flowchart of a possible embodiment of the method according to the present invention carried out with the system as shown in FIG. 1-3.

In the present embodiment, the patient's response with respect to the delivered stimulation is determined based on a patient's feedback on the one or more treatment parameters, provided through a user input device.

For instance, the patient may be affected by Parkinson's disease

In the shown embodiment, the user input device is a smartwatch 200 (FIG. 1) to be worn on the patient's wrist.

For instance, the treatment parameters settings can be judged as optimal by the algorithm upon detecting that the patient's feedback remains positive over a predefined period of time.

If, due to changes in conditions, e.g., physical state of the patient, change in medication, change of seasons, change of lifestyle or the like, the patient is no longer comfortable with the current treatment parameters settings, he/she is allowed to autonomously trigger the algorithm to redefine one or more treatment parameters settings, without the need of visiting a hospital or otherwise involve qualified clinical staff.

By placing the patient and his/her symptoms and priorities at the center while (re)defining the treatment settings, the overall efficacy of the treatment outcome can be significantly enhanced.

Furthermore, the adaptation of the treatment parameter settings can be easily carried out through the algorithm in a patient's home environment, without the need of a hospital visit of requesting the intervention of qualified clinical staff.

The initial settings for the neuromodulator device 100 may be determined referring to a cohort of patients having the same symptoms of the patient subject to treatment (e.g., patients affected by pediatric/adult Dystonia).

For each specific cohort, the treatment outcomes that are considered relevant for the patients are usually put together according to standards that are set by ICHOM (International Consortium for Health Outcomes Measurement).

The treatment outcomes are validated and standardized so that they can be benchmarked.

The determined one or more symptoms and expected outcomes for the patient can be organized according to predefined criteria of priority, e.g., in order of importance.

For instance, a particular patient may be an active speaker and may thus be more interested in having an almost perfect speech control when compared to another subject who is retired and spends his/her days reading books, while speech does not represent an important symptom to control.

In such a case, allowing the patient to carry on performing his/her speaker job significantly improves his/her perceived quality of life and activity of daily living.

The initial settings for the neuromodulation device 100 can be determined according to criteria that are known in the art.

Alternatively, or said initial settings may be defined based on collected data from one or more groups of patients having identical or similar health condition than the patient subject to treatment.

To improve reliability, the collected data preferably relate to a sufficiently large group of patients.

This enhances the possibilities of finding the best settings for the patient subject to treatment.

The algorithm is set to modify the treatment parameters in a predefined range within which the different parameter values are considered safe (e.g., there is no risk of tissue damage).

The algorithm may determine a set of optimal points during the day or during certain conditions (e.g., based on a time of the day and/or medication effectiveness at certain moments of the day) to better satisfy the patient's needs and preferences.

The active delivered therapy during the day may be adapted in a (semi-)permanent way according to the determined set of optimal points.

The one or more treatment parameters may include one or more of:
neuromodulation frequency ranges;
neuromodulation current ranges;
electrode selection,
burst modes and patterns;
level of patient activity in “on” and “off” conditions;
pharmacology;
sleep patterns;
mood patterns, and
weather conditions

The method of the invention actively involves the patient by requiring his/her feedback on the quality of the therapy, i.e., regarding one or more of the treatment parameters.

The patient provides his/her feedback through the user input device 200.

The user input device 200 may be a portable or wearable smart device 200.

As shown in FIG. 1, the input device 200 may be a smartwatch 200 to be worn on the patient's wrist.

In the present embodiment, the smartwatch 200 includes a touch-sensitive display 201 that is configured and adapted to display a user interface including a plurality of selectable options 202.

The user interface can be customized.

The patient can select one or more among the displayed selectable options 202 through a touch input, e.g., tapping.

The selectable options 202 may be displayed on the touch-sensitive display 201 at predefined time intervals (e.g., every half an hour). The selectable options 202 can be displayed as a list.

Alternatively, the selectable options 202 can be displayed as a population of icons 202a on the touch-sensitive display 201 (FIG. 1).

Advantageously, the displayed selectable options 202, e.g., a population of icons 202a, can be color-coded.

As an example, green color can be used for the feedback “I am feeling well”, yellow color can be used for the feedback “I am feeling neutral”, and red color can be used for the feedback “I am feeling bad”.

This provides for a more user-friendly and intuitive environment for the patient.

Additionally or alternatively, a questionnaire may be displayed on touch-sensitive display 201, asking the patient to provide a feedback with respect to one or more specific questions (not shown).

Additionally or alternatively, the user input device may be implemented with means for receiving a vocal input from the patient (not shown).

As an alternative, the user input device can be a (not shown) custom-designed input device.

The method may further include:
collecting information on environmental and/or physiological conditions and/or one or more movements and/or gestures of the patient in real time, and
processing the collected information for use by the algorithm in determining the new settings for the neuromodulation device 100.

Accordingly, accuracy in determining the new settings for the neuromodulation device 100 can be further enhanced.

The environmental condition(s) may include a night/day condition, an active/inactive status of the patient, weather conditions or the like.

The physiological condition(s) may include heart rate, lying/standing/sitting/sleeping conditions or the like.

The information on environmental and/or physiological conditions of the patient can be advantageously collected through sensor means and/or a camera (not shown).

The sensor means and/or camera can be configured and adapted to continually collect information from the patient.

The sensor means and/or camera can be embedded in a portable smart device, e.g., a smartphone (not shown).

Said portable device may be further connected to a device adapted, e.g., for blood pressure or gait/imbalance measurement.

The status of the patient can be represented color-coded in a time scale, such that both the patient and the clinical staff are allowed to intuitively visualize how the patient is feeling and which parameters could be observed and optimized in time.

As an example, over a predefined period of time (e.g., three weeks), the patient may provide a positive feedback (green color) in the morning and a negative feedback (red color) in the evening, meaning that a worsening of his/her conditions occurs at a certain point of the day.

Accordingly, the clinical staff is allowed to investigate at which time of the day the patient's conditions start to worsen, and optimize treatment parameter settings accordingly.

Upon detecting a negative feedback from the patient over a predetermined time period, the algorithm can determine that the patient is in need of hospital assistance.

In such a case, an alert can be triggered in order to notify clinical staff that the patient is in the need of hospital assistance.

Accordingly, the patient can be more promptly summoned for a medical consultation.

On the other hand, when the patient's situation appears under control (e.g., the patient shows a positive feedback most of the time over a predefined time period), the patient is only summoned for a routine hospital checkup, e.g., on an annual basis.

This allows a more efficient organization of clinical staff and an optimization of healthcare resources.

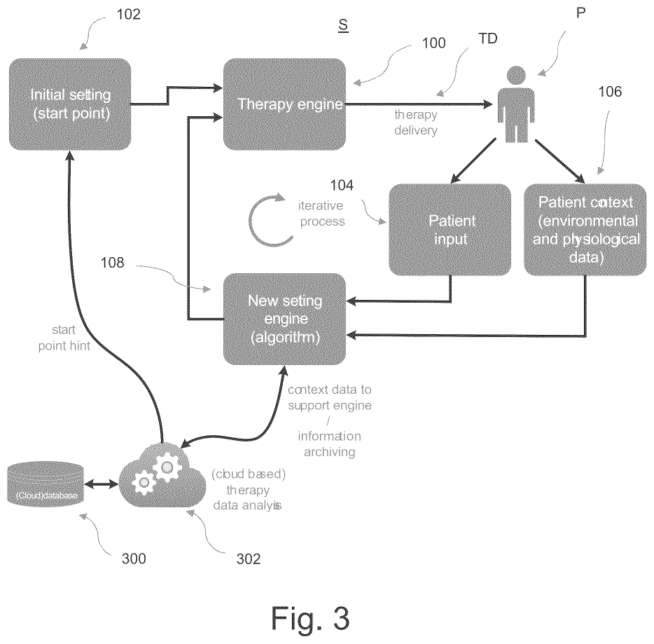

FIG. 3 shows a method for programming a neuromodulator device 100 according to a further embodiment of the invention.

For the sake of conciseness, description of method steps that are equal to the ones described above with regard to FIG. 2 will be omitted.

Basically, the method according to the present embodiment differs from the method shown in FIG. 2 in that it further comprises:
storing, on a database 300 (FIG. 3), data regarding different groups of patients, each of the groups including patients having similar or identical health and therapy conditions;
processing said data according predefined criteria, and
defining the initial settings of the neuromodulation device 100 and the algorithm based on data of a group of patients having similar or identical health and therapy conditions than the patient subject to treatment.

The database 300 is preferably a cloud-based database 300 capable of storing a large number of data.

By storing and analyzing data regarding patients having comparable health conditions it is possible to define the initial settings for the neuromodulator device 100 with improved accuracy and support the algorithm to iterate faster to the endpoint.

In particular, the database 300 is connected to a computing module 302, which is capable to perform a cloud-based therapy data analysis.

The computing module 302 is not only connected to the database 300, but also to the new setting engine 108 and also to the input module for receiving and inputting the initial settings 102 to the neuromodulation device 100.

Privacy of the patient's data is granted in compliance with the regulations and standards of the different countries involved.

One or more filters may be set in the database 300 to allow the operator to more easily and reliably find a group of patients having similar or identical health and therapy conditions than the patient subject to treatment, based on the determined initial health condition of the patient and/or the determined one or more conditions and/or symptoms and/or expected outcomes for the patient.

Advantageously, the algorithm may be further set to predict disease symptoms and/or fluctuations for the patient over time based on the determined patient's response with respect to the delivered stimulation.

In particular, the algorithm may be set to predict symptoms fluctuations over a short temporal scale (e.g., within a day).

Additionally or alternatively, the algorithm may be set to predict disease progression over a longer temporal scale.

Advantageously, the algorithm may be further set to predict disease degeneration for the patient based on detected deviations over time.

The present invention further provides a system programming a neuromodulation device 100.

In the present embodiment, said system includes:
a neuromodulator device 100, e.g., a DBS/DBM therapy delivering device;
a user input device 200 configured and adapted to allow the patient to provide a feedback on one or more treatment parameters, and
processing module.

Specifically, the processing module is configured and arranged to:
(a) determine an initial health condition of the patient (step S (a));
(b) determine one or more conditions and/or symptoms and/or expected outcomes for the patient (step S (b));
(c) determine initial settings for the neuromodulation device 100 including one more treatment parameters, based on the determined initial health condition of the patient and the one or more conditions and/or symptoms and/or expected outcomes, and deliver stimulation to the patient based on the determined initial settings (step S (c));
(d) receive and process a patient's feedback, provided by the patient through the user input device 200, on the one or more treatment parameters (step S (d));
(e) implement an algorithm that is set to modify, based on the patient's feedback, the treatment parameters within a predefined range and determine new settings for the neuromodulation device (100) (step S (e));
(f) repeat steps (d) and (e) at predefined time intervals and/or in response to a user input until obtaining one or more treatment parameters settings that are identified as optimal by the algorithm, based on the patient's feedback (step S (f)), and
(g) maintain the one or more treatment parameters settings identified as optimal by the algorithm and optionally monitor the patient to detect the occurrence of deviations in real time (step S (g)).

The user input device 200 can be a portable or wearable smart device (e.g., a smartwatch) as described above with reference to FIG. 1.

The system may advantageously include a database 300 for storing data regarding groups of patients, each group including patients having similar or identical health and/or therapy conditions (FIG. 3).

Preferably, said database 300 is a cloud-based database 300 (FIG. 3) capable of storing a large number of data.

By using the database 300, preferably a cloud-based database 300, containing many profiles of patients with the same diagnosis and receiving therapy, data analysis on this database can identify high and low performers, that is, patients showing a quick adaptation and patients that, to the contrary, require many iterations.

The identification of patterns that provide an appreciable outcome can be beneficial to those patients showing slow adaptation.

Patient motivation through therapy gamification can be used to improve patient therapy outcomes and/or define outcome-based reimbursement models, where patients showing satisfying treatment outcomes at the end of the year can be compensated with lifestyle rewards or reimbursement of treatment-related costs (e.g., physiotherapy or the like).

This further allows benchmarking the outcomes of the treatment, e.g., to evaluate effectiveness of treatment and efficiency in healthcare resources allocation.

The present invention allows a single physician (e.g., a neurologist) to remotely follow larger numbers of patients when compared to the state of the art.

In more detail, it is expected that a single physician will be able to manage over seven hundred patients, instead of the approximately one hundred that he/she is able to manage in the state of the art.

Further, according to the invention, in-hospital assistance is only provided in case of effective need of the patient.

This allows for a more efficient management of healthcare resources, further than improving the overall organization and coordination of clinical staff.

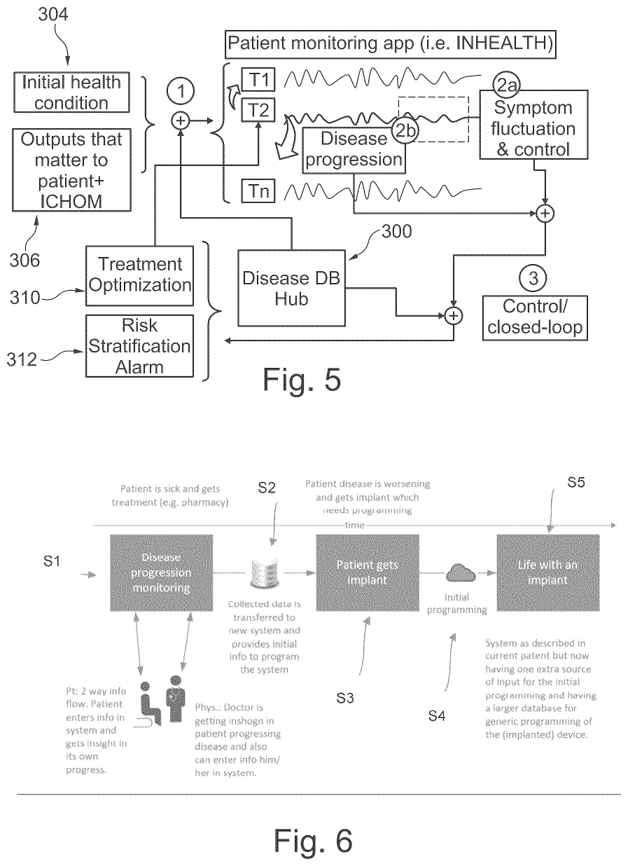

FIG. 5 shows a patient-centered method for optimizing Parkinson's disease (PD) neuromodulation device programming and for improving patient journey and healthcare resources.

In steps S (a), S (b) and S (c), depicted as Part (1), the patient initial health condition 304 and chosen patient data that matter to the patient (=ICHOMs) 306 are inputted into an extensive patient database 300.

The database includes filters that help the doctor tailor the population used to find initial treatment (Tn), starting from treatments T1 to T2 etc..

As further input into the treatment, input from treatment optimization 310 and Risk Stratification Alarm 312 is provided.

There is a treatment personalization based on clusterization of disease stages using population DB+patient profile.

The disease parametrization distinguishes two time scales: (A) Shorter term fluctuation over daily symptoms (both motor & non-motor) and (B) longer term disease progression, describing wearing-off and changes on time on/off range and classifying patient into different patient data (PD) stages (early, moderate and advanced PD)

Part 2 comprises parts 2a and part 2b (which relates to steps d-e):

By means of patient monitoring sensors and active symptom reporting app (i.e. e.g. the app INHEALTH) which is tailored to individual needs (taking of those outcomes and variables that matter to a particular patient): PD individual ups and downs are predicted (including both (part 2a) shorter scale symptoms fluctuations 306 over a predefined and selected treatment (Tn) and (part 2b) disease progression 308 over longer temporal scales)

Further, there is an ups and downs prediction, focused mainly in short term fluctuations and based in forecast prediction and an extraction of individual patterns. The objective is to generate an immediate feedback to the patient, increase patient engagement and empower them. Similar to when a person checks the forecast to better plan his/her routine the patient feedback is not only used to optimize therapy but to generate an individual report of key metrics and a tool to improve the management of the disease.

Another step is to detect the symptom's deviation, over specified thresholds. Further, there can be a predictive maintenance of device.

In Part 3, which relates to steps (f)-(g) and the “control/closed loop”, the control algorithms allow to optimize treatment, to control fluctuations and to extract hidden patterns that may provide earlier signs of key changes within disease. If some important deviations are observed in time, they are studied against the historic data from the patient together with the population database 300 to understand the origin and the best solution. This allows to see if changes are a sign of illness progression and if there is the chance to change the stablished treatment before the symptoms get worst thus preventing patient degeneration.

Also, there is a study of the repetitive deviation, i.e. an automatic control over abnormal symptom fluctuation, alerting physician of a change either originating from altered patient routine or informing that a change of prescribed treatment is required.

There is a need for earlier neurostimulation therapy, which means an earlier use of DBS confers long-term symptomatic benefit. Initial patient monitoring data from the INHEALTH app can recommend physician to prescribe neuromodulation therapy before refractory symptoms are obvious. This will improve patient outcomes for their treatments.

Also, there is a turning point to change a stablished treatment and illness degeneration prediction can be performed.

FIG. 6 shows a flowchart of a possible application of the method and system according to the present invention.

The current idea limits itself in the timeline by starting at the moment of implantation (e.g. stimulation effect). By starting earlier in time collecting data from/by the patient, already:
1) Information can be collected by patient, care giver, doctor, spouse. As can be seen in this step S1, the patient is sick and gets a treatment, e.g. a pharmacological treatment. There can be a two-way information flow, i.e. the patent enters information into the system and also can gain insights into its own progess of recovery or the progress of the treatment.
2) The collected data can be transferred to a new system this will provide initial info to program the system (step S2). Information can be processed in larger setting (patient group) to have more data available. This is part of steps S (a) to S (c) as shown in FIG. 4.
3) Information can be used by patient and doctor to optimize treatment of decisisae (resulting from the analysis and study of the disease progression and the symptom's fluctuation) and e.g. to optimize pharmacological treatment. It also opens the possibility for a physician to study how a change (due to treatment, dosis, patient's circumstances) have an effect on clinical outcomes, being able to take better decisions.
4) A treatment classification can be made.
5) The info is used a starting point at moment of implant, see step S3. This happens, when the patient disease is worsening and the patient needs to get an implant, which then must be programmed (=initial programming in step S4, steps S (d) to S (f) according to FIG. 4 happening here).
6) The gathered info is part of the large database which is build and used for e.g. tuning of the closed loop.

Step S5 is basically that the patient is using the implant in his daily life (see also step S S (f) according to FIG. 4.

Note that the example control and estimation routines included herein can be used with various neuromodulation and/or neurostimulation system configurations. The control methods and routines disclosed herein may be stored as executable instructions in non-transitory memory and may be carried out by the control unit in combination with the various sensors, actuators, and other system hardware in connection with a medical neurostimulation system. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the control unit, where the described actions are carried out by executing the instructions in a system including the various hardware components in combination with a electronic control unit.

### REFERENCE SIGNS USED IN THE FIGURES

100 Neuromodulator device
102 Starting point/initial setting
104 Patient input
106 Patient context
108 New setting engine
200 User input device (wearable smart device, smartwatch)
201 Touch-sensitive display
202 Selectable option
204 Selectable icon
300 Database, cloud-based database
302 Computing Module
304 Initial Health Condition
306 Outputs that matter to patient (=ICHOM)
310 Treatment optimization
312 Risk Stratification Alarm
TD Therapy delivery
P Patient
S System for programming a neuromodulation device
T1 Treatment
T2 Treatment
Tn Treatment
S (a) Step (a)
S (b) Step (b)
S (c) Step (c)
S (d) Step (d)
S (e) Step (e)
S (f) Step (f)
S (g) Step (g)
S1 Step 1
S2 Step 1
S3 Step 3
S4 Step 4
S5 Step 5

1. Method for programming a neuromodulation device, comprising:
(a) determining an initial health condition of a patient;
(b) determining one or more conditions and/or symptoms and/or expected outcomes for the patient;
(c) determining initial settings for the neuromodulation device including one or more treatment parameters based on the determined initial health condition of the patient and/or the one or more conditions and/or symptoms and/or expected outcomes, and delivering stimulation to the patient based on the determined initial settings;
(d) determining a patient's response with respect to the delivered stimulation
(e) implementing an algorithm that is set to modify, based on the determined patient response, the one or more treatment parameters within a predefined range and determine new settings for the neuromodulation device;
(f) repeating steps (d) and (e) at predefined time intervals and/or in response to a user input until obtaining one or more treatment parameters settings that are identified as optimal by the algorithm based on the patient's response to stimulation, and
(g) maintaining the one or more treatment parameters settings identified as optimal by the algorithm.

2. The method according to claim 1,
wherein
the patient's response with respect to the delivered stimulation is determined based on a patient's feedback on the one or more treatment parameters provided through a user input device, the method further comprising monitoring the patient to detect the occurrence of deviations in real time.

3. The method according to claim 2,
wherein
the method further comprises the following steps:
recording brain signals of the patient during stimulation,
the patient's response with respect to the delivered stimulation is determined based on the recorded brain signals.

4. The method according to claim 3,
wherein
the method further comprises the following steps:
detecting one or more movements and/or gestures of the patient during stimulation delivery,
the patient's response with respect to the delivered stimulation is determined based on the detected one or more movements and/or gestures of the patient during stimulation delivery.

5. The method according to claim 1,
wherein
the method further comprises the step of organizing the determined one or more conditions and/or symptoms and/or expected outcomes for the patient according to predefined criteria of priority.

6. The method according to claim 1,
wherein
the initial settings for the neuromodulation device are determined based on collected data from one or more groups of patients having identical or similar health conditions than the patient subject to treatment.

7. The method according to claim 1,
wherein
the one or more treatment parameters include one or more of:
neuromodulation frequency ranges;
neuromodulation current or voltage ranges;
electrode selection;
burst modes and patterns;
level of patient activity in “on” and “off” conditions;
pharmacology;
sleep patterns;
mood patterns;
environmental and/or weather conditions that may affect a health condition of the patient, and/or
one or more anticipated activities of the patient.

8. The method according to claim 2,
wherein
the user input device is a portable or wearable device.

9. The method according to claim 8,
wherein
the user input device includes a touch-sensitive display that is configured and adapted to display a user interface including a plurality of selectable options, and allow selection of one or more of the displayed selectable options through a touch input.

10. The method according to claim 9,
wherein
the method further comprises the step of displaying a plurality of selectable options on the touch-sensitive display at predefined time intervals, wherein
the displayed plurality of selectable options is color-coded.

11. (canceled)

12. The method according to claim 8,
wherein
the user input device further includes means for receiving a vocal input from the patient.

13. The method according to claim 1,
wherein
the method further comprises the following steps:
collecting information on environmental and/or physiological conditions and/or one or more movements and/or gestures of the patient in real time, and
processing the collected information for use by the algorithm in determining the new settings for the neuromodulation device.

14. The method according to claim 13,
wherein
said information on environmental and/or physiological conditions and/or one or more movements and/or gestures of the patient is collected through sensor means and/or a camera and/or a microphone or voice command module, wherein
said sensor means and/or camera are embedded in a portable device of the patient.

15. (canceled)

16. The method according to claim 1,
wherein
the method further comprises the following steps:
storing, on a database, data regarding different groups of patients, each of the groups including patients having similar or identical health and therapy conditions;
processing said data according predefined criteria, and
defining the initial settings of the neuromodulation device and the algorithm based on data of a group of patients having similar or identical health and therapy conditions than the patient subject to treatment.

17. The method according to claim 16,
wherein
the method further comprises the following step:
setting one or more filters in said database to allow an operator to find a group of patients having similar or identical health and therapy conditions than the patient subject to treatment based on the determined initial health condition of the patient and/or the determined one or more conditions and/or symptoms and/or expected outcomes for the patient.

18. The method according to claim 1,
wherein
the algorithm is further set to:
predict disease symptoms and/or fluctuations for the patient over time based on the determined patient's response with respect to the delivered stimulation,
wherein predicting disease symptoms and fluctuations over time includes:
predicting disease symptoms fluctuations over a short temporal scale, and/or
predicting disease progression over a longer temporal scale,
wherein the algorithm is further set to:
predict disease degeneration for the patient based on detected deviations over time.

19. (canceled)

20. (canceled)

21. System for programming a neuromodulation device, comprising:
a neuromodulator device, and
processing module that is configured and adapted to:
(a) determine an initial health condition of the patient;
(b) determine one or more conditions and/or symptoms and/or expected outcomes for the patient;
(c) determine initial settings for the neuromodulation device including one more treatment parameters based on the determined initial health condition of the patient and/or the one or more conditions and/or symptoms and/or expected outcomes, and deliver stimulation to the patient based on the determined initial settings;
(d) determine a patient's response with respect to the delivered stimulation;
(e) implement an algorithm that is set to modify, based on the determined patient's response, the one or more treatment parameters within a predefined range and determine new settings for the neuromodulation device;
(f) repeat steps (d) and (e) at predefined time intervals and/or in response to a user input until obtaining one or more treatment parameters settings that are identified as optimal by the algorithm, based on the patient's response to stimulation, and
(g) maintain the one or more treatment parameters settings identified as optimal by the algorithm.

22. The system according to claim 21,
wherein
the system further comprises:
a user input device configured and adapted to allow a patient to provide feedback on one or more treatment parameters,
wherein the processing module is further configured and adapted to:
receive and process a patient's feedback on the one or more treatment parameters, provided through the user input device.

23. The system according to claim 22,
wherein
the user input device is a portable or wearable device, including a touch-sensitive display that is configured and adapted to display a user interface including a plurality of selectable options, and allow selection of one or more of the displayed selectable options through a touch input.

24. The system according to claim 21,
wherein
the system further comprises a database, for storing data regarding groups of patients, each group including patients having similar or identical health and/or therapy conditions.
